(19) United States
(12) Patent Application Publication
(10) Pub. No.: US 2022/0347169 A1
(43) Pub. Date: Nov. 3, 2022

(54) USING GENO- OR PHENOTYPING TO ADJUST LSD DOSING

(71) Applicant: Universitätsspital Basel, Basel (CH)

(72) Inventors: Matthias Emanuel LIECHTI, Oberwil (CH); Patrick Raphael VIZELI, Kaiseraugst (CH)

(73) Assignee: Universitätsspital Basel, Basel (CH)

(21) Appl. No.: 17/869,896

(22) Filed: Jul. 21, 2022

Related U.S. Application Data
(63) Continuation of application No. 17/685,138, filed on Mar. 2, 2022.
(60) Provisional application No. 63/157,684, filed on Mar. 6, 2021.

(51) Int. Cl. A61K 31/4745; C12Q 1/6876

(52) U.S. Cl. CPC A61K 31/4745; C12Q 1/6876; C12Q 2600/106; C12Q 2600/156

(57) ABSTRACT

A method of dosing LSD in treating patients, by assessing genetic characteristics in the patient by identifying polymorphisms of CYP2D6 before use of a composition chosen from the group consisting of LSD, analogs thereof, derivatives thereof, and salts thereof, administering the composition to the patient based on the patient genetics, wherein a 50% dose is administered in a patient with non-functional CYP2D6 compared to a dose in functional CYP2D6 individuals, and producing maximum positive subjective acute effects in the patient and/or reducing anxiety and negative effects. A method of determining a preferred dose of LSD.

### GRANT INFORMATION

Research in this application was supported in part by grants from the Swiss National Science Foundation (grant no. 320030_170249 and 320036_185111).

### BACKGROUND OF THE INVENTION

### 1. Technical Field

The present inventions relates to a method of genetic testing and adjusting the dose and predicting effects of LSD used in humans in medical treatments.

### 2. Background Art

Lysergic acid diethylamide (LSD) can be used to assist psychotherapy for many indications including anxiety, depression, addiction, personality disorder, and others and can also be used to treat other disorders such as cluster headache, migraine, and others (Hintzen & Passie, 2010; Liechti, 2017; Nichols, 2016; Passie et al., 2008). LSD targets the 5HT2A receptor, which is a serotonin receptor. Effects of LSD can include altered thoughts, feelings, awareness of surroundings, dilated pupils, increased blood pressure, and increased body temperature.

Doses commonly used in LSD-assisted treatment/psychotherapy are 100-200 μg. A dose of 100 μg produced subjective effects in humans lasting (mean±SD) 8.5±2.0 hours (range: 5.3-12.8 hour) in one representative study (Holze et al., 2019). In other studies, LSD effects similarly lasted 8.2±2.1 hours (range: 5-14 hours) after administration of a 100 μg dose and 11.6±1.7 hours (range: 7-19.5 hours) after administration of a 200 μg dose (Dolder et al., 2017b).

The acute subjective effects of LSD are mostly positive in most humans (Holze et al., 2021b; Schmid et al., 2015). However, there are also negative subjective effects (anxiety) of LSD in many humans depending on the dose of LSD used, the setting (environment), and the set which includes personality traits of the person using LSD but also possibly other factors such as the metabolic enzymes present in a person and individual characteristic of the sites of action of LSD (serotonin receptors).

The risk of acute negative psychological effects is the main problem of use of psychedelic substances in humans. Anxiety when occurring in LSD-assisted psychotherapy may become a significant problem for both the patient and treating physician. In addition to being highly distressing to the patient, acute anxiety has been linked to a non-favorable long-term outcome in patients with depression (Roseman et al., 2017). Furthermore, anxiety reactions during psychedelic-assisted therapy may require additional supervision, greater engagement of therapists, prolonged sessions, and acute psychological and also pharmacological interventions. Thus, the primary safety concerns relate to psychological rather than somatic adverse effects (Nichols, 2016; Nichols & Grob, 2018). The induction of an overall positive acute response to the psychedelic is critical because several studies showed that a more positive experience is predictive of a greater therapeutic long-term effect of the psychedelic (Garcia-Romeu et al., 2014; Griffiths et al., 2016; Ross et al., 2016). Even in healthy subjects, positive acute responses to psychedelics including LSD has been shown to be linked to more positive long-term effects on well-being (Griffiths et al., 2008; Schmid & Liechti, 2018).

Moderate anticipatory anxiety is common at the beginning of the onset of a drug's effects (Studerus et al., 2012). In a study in sixteen healthy humans, after administration of 200 μg of LSD marked anxiety was observed in two subjects. This anxiety was related to fear of loss of thought control, disembodiment, and loss of self (Schmid et al., 2015) and was similarly described for psilocybin (Hasler et al., 2004). Bad drug effects (50% or more on a 0-100% scale at any time point after drug administration) were noted in 9 of 16 subjects (56%) after a high dose of 200 μg of LSD and in 3 of 24 subjects (12.5%) after a moderate 100 μg dose of LSD (Dolder et al., 2017a). Similarly, another study reported transient bad drug effects in 7 of 24 subjects (29%) after administration of 100 μg of LSD (Holze et al., 2019a). Although, these negative subjective drug effects were transient and occurred in subjects who all also reported good drug effects at other or/and similar time points, negative responses are an issue.

One solution to address negative drug effects is to generally reduce the dose of the psychedelic but this also reduces at least in part the drug efficacy and a dose reduction may be needed only in some but not other patients.

While pharmacogenetic approaches have been used for several medications, no information on the pharmacogenetics of LSD has been available so far that would allow dose adjustment for LSD. There is no direction in the prior art as to how pharmacogenetics would be applied.

Independently, in vitro metabolic studies indicate that several cytochrome P450 (CYP) isoforms (e.g., CYP2D6, CYP1A2, CYP2C9) are involved in the metabolism of LSD but in vivo data is missing as well as any application of such studies to altered dosing of LSD.

The psychedelic effects of LSD are primarily mediated by the agonism at the 5-hydroxytryptamine (5-HT) 2A receptor (5HTR2A) (Holze et al., 2021b; Kraehenmann et al., 2017). However, LSD binding acts also as a partial agonist to other 5-HT receptors such as 5HTR1A, 5HTR2B and 5HTR2C (Rickli et al., 2016).

There remains a need for accurate dosing of LSD as well as personalized dosing of LSD to reduce adverse drug effects.

### SUMMARY OF THE INVENTION

The present invention provides for a method of dosing LSD in treating patients, by assessing genetic characteristics in the patient by identifying polymorphisms of CYP2D6 before use of a composition chosen from the group consisting of LSD, analogs thereof, derivatives thereof, and salts thereof, administering the composition to the patient based on the patient genetics, wherein a 50% dose is administered in a patient with non-functional CYP2D6 compared to a dose in functional CYP2D6 individuals, and producing maximum positive subjective acute effects in the patient and/or reducing anxiety and negative effects.

The present invention provides for a method of determining a preferred dose of LSD, by determining metabolic or/and genetic markers in a patient by assessing CYP2D6 activity in the patient, adjusting a dose of a composition chosen from the group consisting of LSD, analogs thereof, derivatives thereof, and salts thereof based on metabolic and genetic activity (pharmacogenetics), wherein if CYP26D activity is poor or not present, the dose is adjusted to 50% of a dose with functional CYP26D, administering the dose of the composition to the patient, and producing maximum positive subjective acute effects in the patient and/or reducing anxiety and negative effects.

### DESCRIPTION OF THE DRAWINGS

Advantages of the present invention are readily appreciated as the same becomes better understood by reference to the following detailed description when considered in connection with the accompanying drawings wherein:

FIG. 1 is a graph of the modeled LSD plasma concentration-time curves over 24 hours after LSD administration to subjects with genetically determined non-functional (red) or functional (blue) CYP2D6 enzymes;

FIG. 2 shows a graph of a linear regression model of body weight (kg) of the participants versus LSD plasma exposure expressed as infinite area-under-the-curve (AUC∞) (z-score);

FIG. 3 shows a table of the effects of CYP2D6 on the LSD pharmacokinetics;

FIG. 4 shows a table of the effects of CYP2D6 on the pharmacokinetics of the main LSD metabolite O-H-LSD;

FIG. 5 shows a table of the effects of CYP2D6 on the subjective and autonomic effects of LSD;

FIG. 6 shows a table of the effects of CYP2D6 on the acute alterations of the mind induced by LSD;

FIG. 7 shows a table of the effects of the HTR1B rs6296 genotype on the effects of LSD;

FIG. 8 shows a table of the effects of the HTR1A rs6295 genotype on the effects of LSD;

FIG. 9 shows a table of the effects of the HTR2A rs6313 on the effects of LSD;

FIG. 10 shows a table of the example study population;

FIG. 11 shows a table of the allele frequency and classification of CYP2D6;

FIG. 12 shows a table of the allele frequency and activity score of CYP2C19 genotypes;

FIG. 13 shows a table of the single nucleotide polymorphism frequencies within the tested genotypes;

FIG. 14 shows a table of the subjective effects of LSD;

FIG. 15 shows a table of the autonomic effects of LSD;

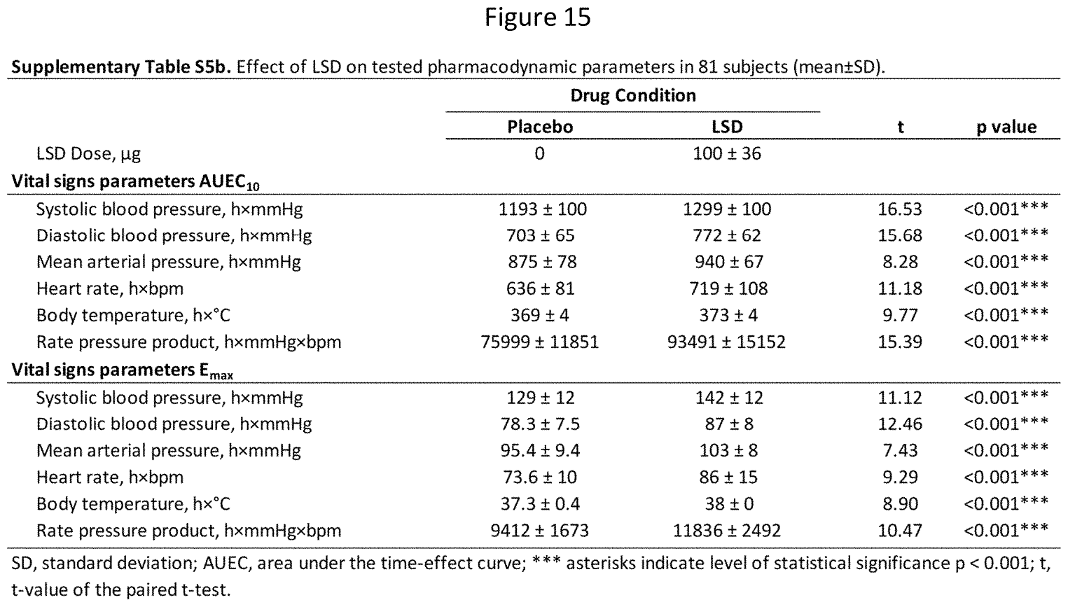

FIG. 16 shows a table of the alterations of mind induced by LSD;

FIG. 17 shows a table of the effects of the CYP2D6 activity score on LSD pharmacokinetics;

FIG. 18 shows a table of the effects of the CYP2C19 activity score on the LSD pharmacokinetics;

FIG. 19 shows a table of the effects of the CYP1A2 genotype on the pharmacokinetics of LSD;

FIG. 20 shows a table of the effects of the CYP2C19 genotype on the pharmacokinetics of LSD

FIG. 21 shows a table of CYP2B6 rs3745274 on the pharmacokinetics of LSD; and

FIG. 22 shows a table of the CYP1A2 rs762551 genotype on the pharmacokinetics of LSD.

### DETAILED DESCRIPTION OF THE INVENTION

The present invention provides for methods of using pharmacogenetics to better define a dose of LSD in patients (humans) before administration. The methods herein provide a personalized treatment for patients with LSD.

More specifically, the present invention provides for a method of dosing LSD in treating patients, by assessing genetic patient characteristics before LSD use, administering LSD to the patient at a dose based on the patient genetics, a use to train therapists, or any other legal controlled setting in healthy subjects, and producing maximum positive subjective acute effects in the subject. The method can also be used to reduce anxiety and negative effects of LSD.

An additional goal of the present invention is to maximize efficacy of LSD administration or at least be able to efficaciously treat a diverse population of patients while maintaining safety and minimizing adverse effects.

While LSD is referred to throughout the application, it should be understood that analogs thereof, derivatives thereof, or salts thereof can also be used. The invention allows for dose-optimization of LSD analogs if they are partly metabolized by CYP2D6 similar to LSD.

After the patient's genetic characteristics are assessed, they can be used to adjust the dose in patients with genetic profiles predicting a greater or more adverse response to LSD. Specifically, a reduced activity of enzymes involved in the metabolism of LSD or genetic alterations in the pharmacological targets of LSD can be determined and the dose of LSD adjusted. Preferably, the LSD is administered in a therapeutic situation or in a legal controlled situation in healthy subjects including but not limited to a clinical study.

The present invention used psychometric, pharmacokinetic, and genetic data from a large sample of controlled LSD administrations to humans to determine the pharmacogenetics of both the key metabolizing enzymes and the target receptors of LSD with regards to its acute effects and thereby newly providing data and specific instructions to adjust LSD doses based on genetics.

Additional variables including age, personality, treatment setting, past psychedelic experience of the person, and others can also be useful to determine the right dose of LSD in addition to the method used herein but are not part of the present invention.

The invention uses data from a clinical study to examine the influence of genetic polymorphisms within CYP genes on the pharmacokinetics and acute effects of LSD in healthy subjects. The study has been published after filing the provisional patent application (Vizeli et al., 2021). LSD potently binds to 5HTR2A and 1A/B receptors and its psychedelic effects dependent on 5HTR2A activation and can therefore be moderated by genetic variations in these receptor genes. The invention therefore identified common genetic variants of CYPs (CYP2D6, CYP1A2, CYP2C9, CYP2C19, CYP2B6) and serotonin receptors (5HTR1A, 5HTR1B, and 5HTR2A) in 81 healthy subjects pooled from four randomized, placebo-controlled, double-blind phase 1 studies to derive the data needed for the present invention.

The study showed that genetically determined CYP2D6 functionality significantly influenced the pharmacokinetics of LSD. Individuals with no functional CYP2D6 alleles (poor metabolizers) had longer LSD half-life values and approximately 75% higher parental drug and main metabolite 2-oxo-3-hydroxy LSD (O-H-LSD) area under the curve blood plasma concentrations compared to individuals who were carriers of functional CYP2D6 alleles. Non-functional CYP2D6 metabolizers also showed greater alterations of the mind and longer subjective effect durations in response to LSD compared with functional CYP2D6 metabolizers. No effect on the pharmacokinetics or acute effects of LSD were observed with other CYPs.

Variants in the target receptors of LSD also weakly moderated the acute effects of LSD on the 5D-ASC scale. Specifically, carriers of two HTR2A rs6313 A alleles showed lower alterations of the mind (total 5D-ASC score and anxious ego-dissolution) than G allele carriers. Homozygous carriers of the HTR1A rs6295 G allele reported lower total 5D-ASC, Visionary Restructuralization, and Blissful State ratings compared to carriers of a C allele.

Taken together the present invention shows that genetic polymorphisms influence LSD effects in humans. Specifically, the genetic polymorphisms of CYP2D6 had a significant influence on the pharmacokinetics and the subjective effects of LSD. It can therefore be used to define the dose of LSD based on genetic testing and interpretation of the findings using the presently developed invention.

The dose of LSD can be 50% in patients with non-functional CYP2D6 compared to a dose in functional CYP2D6 individuals (i.e., 100 μg compared to 200 μg).

Therefore, the present invention provides for a method of determining a preferred dose of LSD, by determining metabolic and genetic markers (such as by assessing CYP2D6 activity and/or assessing 5HTR1A rs6295 and 5HTR2A rs6313 genotypes) in a patient, adjusting a dose of LSD based on the genetically or otherwise determined metabolic activity and genetics of the pharmacological target receptors (i.e. the CYP2D6 activity, and/or 5HTR1A rs6295 and 5HTR2A rs6313 genotypes), and administering the dose of LSD to the patient. The metabolic activity can be related to enzymatic digestion. The pharmacological activity can be related to activation or binding to receptors (primary sites of action such as 5-HT1 and 5-HT2 and others). The genotype of the genes coding for the receptors can increase or decrease binding, psychedelic effect, actual efficacy, etc. By understanding these pharmacogenetic effects, dosing can be adjusted to tailor those effects appropriately for an individual patient or a well-defined group of patients sharing genetic signatures.

The present invention also provides for a method of determining a dose of LSD based on an assessment of the presence of CYP2D6 inhibitors by assessing concomitant medications of CYP2D6 inhibition potential in a patient, assessing CYP2D6 activity in a patient, administering LSD to the patient, and producing maximum positive subjective acute effects in the patient and/or reducing anxiety and negative effects. Some patients are treated with serotonin reuptake inhibitors that can act as CYP2D6 inhibitors, such as fluoxetine or paroxetine. Such individuals can also have reduced CYP2D6 activity due to genetics. Therefore, CYP2D6 inhibitors can be stopped before LSD treatment begins so that the enzyme can regenerate (up to two weeks), or the dose of LSD can be adjusted to be reduced in the presence of CYP2D6 inhibitors.

The invention further shows that common mutations in the 5-HT receptor genes influence the acute alterations of the mind induced by LSD. This pharmacogenetic effect can be considered in LSD research and LSD-assisted psychotherapy by using the present data and instructions.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, sublingual, subcutaneously, transcutaneously or parenterally including intravenous, intramuscular, and intranasal administration and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

The present invention was developed based on data from a pooled analysis of clinical studies presented herein in detail. This study has been published after filing the provisional patent application (Vizeli et al., 2021).

Background of the Study

Despite its widespread use, the metabolism of LSD is not fully understood. Two recent in vitro studies showed an involvement of cytochrome P450 enzymes (CYPs) in the metabolism of LSD (Luethi et al., 2019; Wagmann et al., 2019). One study using human liver microsomes showed that CYP2D6, 3A4, and 2E1 contribute to the N-demethylation of LSD to 6-nor-LSD (Nor-LSD), while CYP2C9, CYP1A2, CYP2E1, and CYP3A4 take part in the formation of the main metabolite 2-oxo-3-hydroxy-LSD (O-H-LSD) (Luethi et al., 2019). Another study using human liver S9 fraction found that CYP2C19 and 3A4 were involved in the formation of Nor-LSD and CYP1A2 and CYP3A4 contributed to the hydroxylation of LSD (Wagmann et al., 2019).

Some CYPs (i.e., CYP2D6, CYP1A2, CYP2C9, CYP2C19) have common functional genetic polymorphisms which result in different phenotypes (Gaedigk, 2013; Hicks et al., 2015; Hicks et al., 2013; Preissner et al., 2013; Sachse et al., 1997; Sachse et al., 1999). Mostly, CYP2D6 exhibits several phenotypes from poor metabolizers (PMs, 5-10% in Caucasian) to ultra-rapid metabolizers (UMs, 3-5%) with different underlying genotypes (Sachse et al., 1997). Genetic variants of LSD-metabolizing CYPs, in particular CYP2D6 (Luethi et al., 2019), could influence the pharmacokinetics of LSD and also its acute effects that are closely linked to the plasma concentration-time curve of LSD within an individual (Holze et al., 2019; Holze et al., 2021a; Holze et al., 2021b). CYP2D6 genotype has also previously been shown to influence the pharmacokinetics of 3,4-methylenedioxymethamphetamine (MDMA) (Schmid et al., 2016; Vizeli et al., 2017), a substance also used for substance-assisted psychotherapy (Schmid et al., 2021).

This analysis as part of the present invention investigated the influence of prominent genetic polymorphisms of important CYPs (CYP2D6, CYP1A2, CYP2C9, CYP2C19, CYP2B6) on the pharmacokinetic parameters of LSD and its acute subjective effects.

The quality and extent of the subjective effects of psychedelics are of particular interest because more intense and more positive acute psychedelic effects are thought to predict long-term therapeutic outcome in patients treated in psychedelic-assisted therapy (Griffiths et al., 2016; Roseman et al., 2017; Ross et al., 2016) and also positive long-term effects in healthy subjects (Griffiths et al., 2008; Schmid & Liechti, 2018).

LSD very potently binds to and acts as a partial agonist at several 5-HT receptors including the 5HTR1A, 5HTR2B and 5HTR2C subtype (Eshleman et al., 2018; Kim et al., 2020; Rickli et al., 2016; Wacker et al., 2017). However, the various psychedelic effects of LSD are thought to be primarily mediated by the agonism at the 5HTR2A (Holze et al., 2021b; Kraehenmann et al., 2017; Preller et al., 2017). Variations in genes that encode key targets in the 5-HT systems could moderate the acute effects of LSD.

There has so far been no data on the pharmacogenetics of LSD or other psychedelics.

However, the single nucleotide polymorphism (SNP) HTR2A rs6313 weakly influenced MDMA effects such as “good drug effect”, “drug liking”, or “closeness to others” (Vizeli et al., 2019).

Additionally, the C allele of the rs6313 SNP is associated with lower expression and was found to be associated with suicide, a lower ability to adopt the point of view of others, greater anxiety when observing pain, and communication problems (Ghasemi et al., 2018; Gong et al., 2015; Polesskaya et al., 2006).

Further, the rs6295 SNP of the HTR1A gene, which encodes the 5HTR1A, may play a role in substance use disorder (Huang et al., 2004). Female homozygous carriers of the G allele of the rs6295 who suffered from major depressive disorder benefited more from treatment with a 5-HT reuptake inhibitor compared with carriers of the C allele (Houston et al., 2012).

The rs6296 SNP of HTR1B, which encodes the 5HTR1B receptor, was found to influence childhood aggressive behavior. Individuals who were homozygous for the C-allele were more aggressive than those who carried the G allele (Hakulinen et al., 2013). The 5-HT receptors are one of the most researched pharmacological targets of psychoactive drugs. However, this is the first information on the pharmacogenetics of a classic serotonergic psychedelic substance in humans.

It was tested whether genetic polymorphism in key metabolic enzymes involved in the breakdown of LSD including CYP2D6, CYP1A2, CYP2C9, CYP2C19 and CYP2B6 or in key targets of LSD including HTR1A, HTR1B, and HTR2A would moderate the pharmacokinetics of acute effects of LSD in healthy subjects.

While LSD was used to develop the present invention, LSD analogs or derivates may also be used if CYP2D6 contributes to the metabolism as in LSD.

Additionally, because all psychedelics act primarily via 5-HT1/2 receptors, HTR1A, HTR1B, and HTR2A genetics can similarly be used for pharmacogenetic dosing of any other psychedelics such as psilocybin, mescaline, dimethyltryptamine (DMT) or others.

Methods

Study Design

This was a pooled analysis of four phase 1 studies that each used a randomized, double-blind, placebo-controlled, crossover design and were conducted in the same laboratory (Dolder et al., 2017b; Holze et al., 2021b; Holze et al., 2020; Schmid et al., 2015).

The studies were all registered at ClinicalTrials.gov (Study 1: NCT01878942, Study 2: NCT02308969, Study 3: NCT03019822, and Study 4: NCT03321136). The studies included a total of 84 healthy subjects. Study 1 (Schmid et al., 2015) and Study 4 (Holze et al., 2021b) each included 16 subjects, Study 2 included 24 subjects (Dolder et al., 2017b). Study 3 included 29 subjects (Holze et al., 2020).

In study 1, each subject received a single dose of 200 μg LSD or placebo. In Study 2 and 3, each subject received a single dose of 100 μg LSD or placebo. In study 4, each subject received 25, 50, 100, and 200, and 200 μg LSD+40 mg ketanserin (a 5-HT2A antagonist). For this pooled analysis, the mean data was used of the four LSD doses used within the same subject in Study 4. The 200 μg LSD+40 mg ketanserin condition was used for the pharmacokinetic analysis but not for the analysis of the effect of LSD.

All studies were approved by the local ethics committee. and were conducted in accordance with the Declaration of Helsinki. The use of LSD was authorized by the Swiss Federal Office for Public Health (Bundesamt für Gesundheit), Bern, Switzerland. Written informed consent was obtained from all of the participants. All of the subjects were paid for their participation.

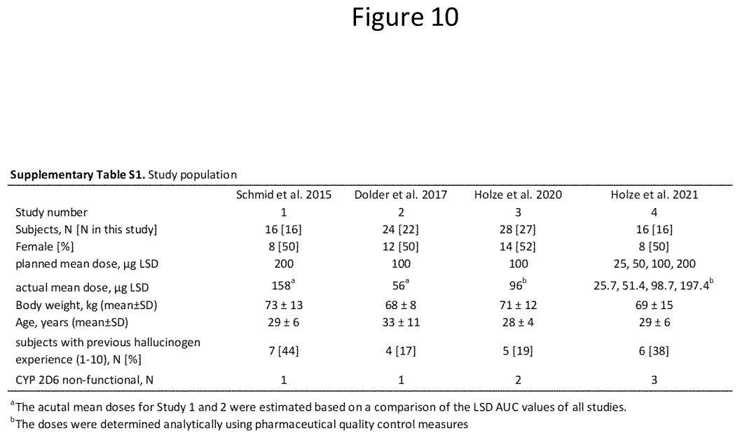

The washout periods between doses were 7 days for Study 1 and 2 and 10 days for Study 3 and 4. Test sessions were conducted in a quiet hospital research ward with no more than one research subject present per session. The subjects were under constant supervision while they experienced acute drug effects. The participants were comfortably lying in hospital beds and were mostly listening to music and not engaging in physical activities. LSD was given after a standardized small breakfast in the morning. A detailed overview of the included studies is shown in FIG. 10 (Table S1).

Subjects

A total of 85 healthy subjects of European descent and 25-60 years old (mean±SD=30±8 years) were mostly recruited from the University of Basel campus and participated in the study. One participant quit before the final LSD session, one participant stopped participation before the first test session, and two participants did not give consent for genotyping, resulting in a final dataset for the analysis of 81 subjects (41 women). The subjects' mean±SD body weight was 70±12 kg (range: 50-98 kg). Participants who were younger than 25 years old were excluded from participating in the study because of the higher incidence of psychotic disorders and because younger ages have been associated with more anxious reactions to hallucinogens (Studerus et al., 2012). The exclusion criteria included a history of psychiatric disorders, physical illness, tobacco smoking (>10 cigarettes/day), a lifetime history of illicit drug use more than 10 times (with the exception of past cannabis use), illicit drug use within the past 2 months, and illicit drug use during the study, determined by urine tests that were conducted before the test sessions. Twenty-two subjects had prior hallucinogenic drug experiences, of which 16 subjects had previously used lysergic acid diethylamide (1-3 times), five subjects had previously used psilocybin (1-3 times), and one subject had previously used dimethyltryptamine (4 times), mescaline (1 time), and salvia divinorum (3 times).

Study Drug

LSD base (Lipomed AG, Arlesheim, Switzerland) was prepared to be taken orally as gelatin capsules (Dolder et al., 2017b; Schmid et al., 2015) in Studies 1 and 2 or as a drinking solution in 96% ethanol in Studies 3 and 4 (Holze et al., 2021b; Holze et al., 2020).

The doses used in each study are shown in Table S1. Content uniformity and long-term stability data was available for the doses used in studies 3-4 (Holze et al., 2019; Holze et al., 2021b; Holze et al., 2020) and the exact actual mean doses of LSD base administered are shown in FIG. 10 (Table S1).

The planned mean doses used in studies 1 and 2 were later detected to be lower and the actual doses used were estimated based on the comparison of area-under-the-curve (AUC) values from Studies 1 and 2 with AUC values from of Studies 3 and 4 (Holze et al., 2019). The doses were not adjusted for body weight or sex.

Pharmacokinetic Analyses

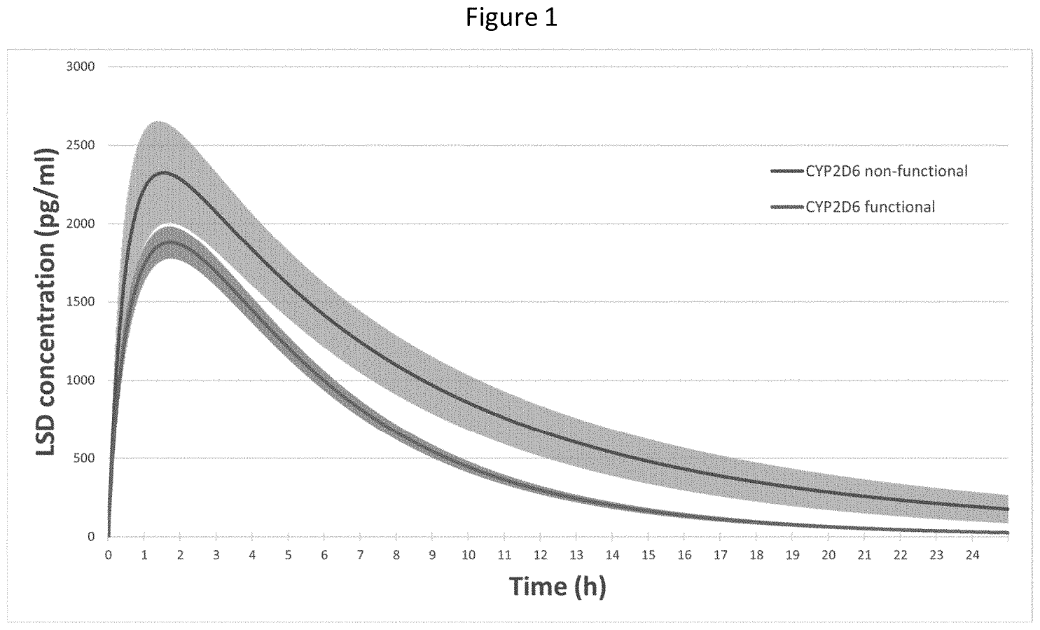

Pharmacokinetic parameters were calculated using non-compartmental analysis in Phoenix WinNonlin 6.4 (Certara, Princeton, N.J., USA). Eₘₐₓ values were obtained directly from the observed data. AUC and AUEC values were calculated using the linear-log trapezoidal method. AUC values were calculated up to the last measured concentration in all studies (AUC₁₀) and extrapolated to infinity (AUC∞). Additionally, a one-compartment model with first-order input, first-order elimination, and no lag time was used in Phoenix WinNonlin 6.4. to compare the pharmacokinetics of LSD in functional and non-functional CYP2D6 groups and to illustrate the LSD concentrations over time (FIG. 1) after a dose of 100 μg LSD base. This analysis included the data from all 81 subjects. For study 4, only the 100 μg dose was included. The onset, offset, and duration of the subjective response were determined using the VAS “any drug effect”-time curve, with 10% of the individual maximal response as the threshold, in Phoenix WinNonlin.

Physiological Effects

Blood pressure, heart rate, and body temperature were assessed repeatedly before and 0, 0.5, 1, 1.5, 2, 2.5, 3, 4, 5, 6, 7, 8, 9, and 10 h after LSD or placebo administration. Systolic and diastolic blood pressure and heart rate were measured using an automatic oscillometric device (OMRON Healthcare Europe NA, Hoofddorp, Netherlands). The measurements were performed in duplicate at an interval of 1 minute and after a resting time of at least 5 minutes. The averages were calculated for the analysis. Mean arterial pressure (MAP) was calculated as diastolic blood pressure+(systolic blood pressure−diastolic blood pressure)/3. The rate pressure product (RPP) was calculated as systolic blood pressure×heart rate. Core (tympanic) temperature was measured using a Genius 2 ear thermometer (Tyco Healthcare Group LP, Watertown, N.Y., USA).

Subjective Effects

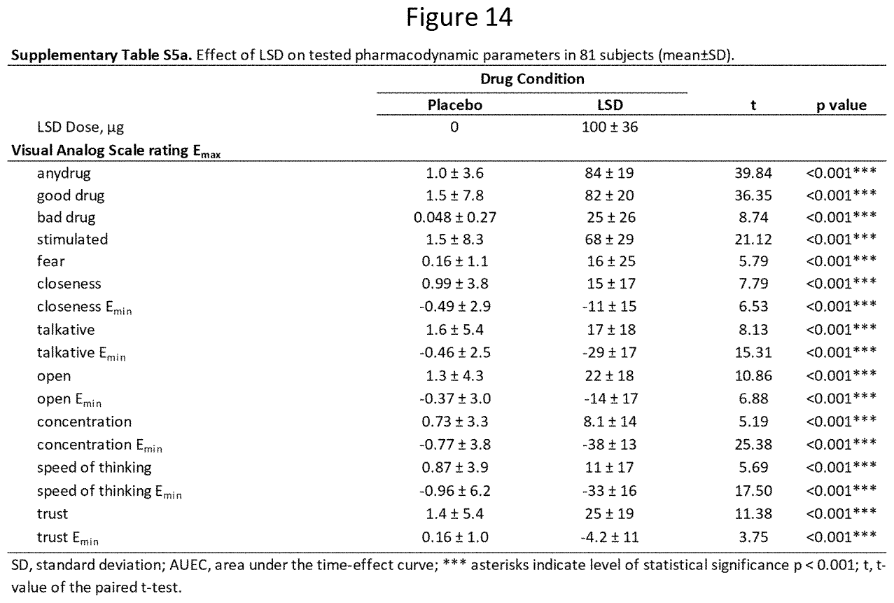

The Visual Analog Scales (VASs, FIG. 14, Table S5) were presented as 100 mm horizontal lines (0-100%), marked from “not at all” on the left to “extremely” on the right. Subjective effects like “closeness”, “talkative”, “open”, “concentration”, “speed of thinking”, and “trust” were bidirectional (±50 mm). The VASs were applied before and 0, 0.5, 1, 1.5, 2, 2.5, 3, 4, 5, 6, 7, 8, 9 and 10 h after LSD or placebo administration.

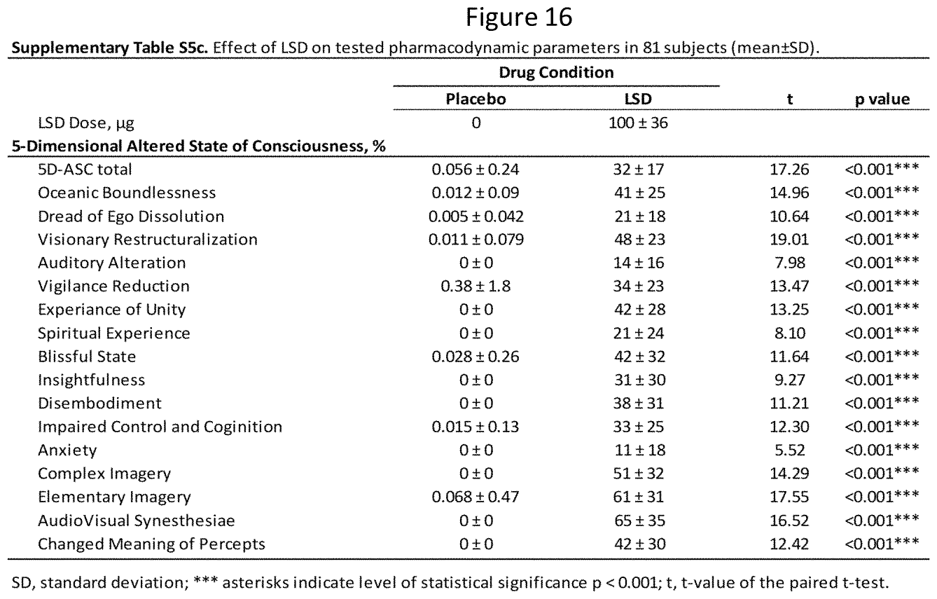

The 5 Dimensions of Altered States of Consciousness (5D-ASC) scale (Dittrich, 1998; Studerus et al., 2010) was administered at the end of the acute drug effects to retrospectively rate peak drug responses. The main subscales describing alterations of consciousness are Oceanic Boundlessness (OB), Anxious of Ego Dissolution (AED), Visionary Restructuralization (VR) (FIG. 16).

Genotyping

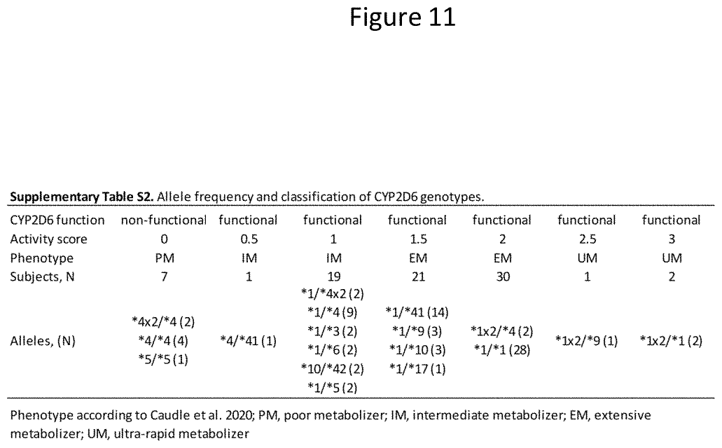

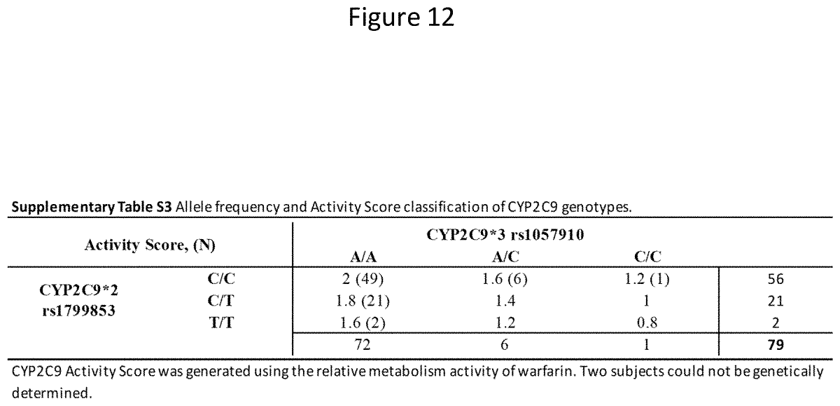

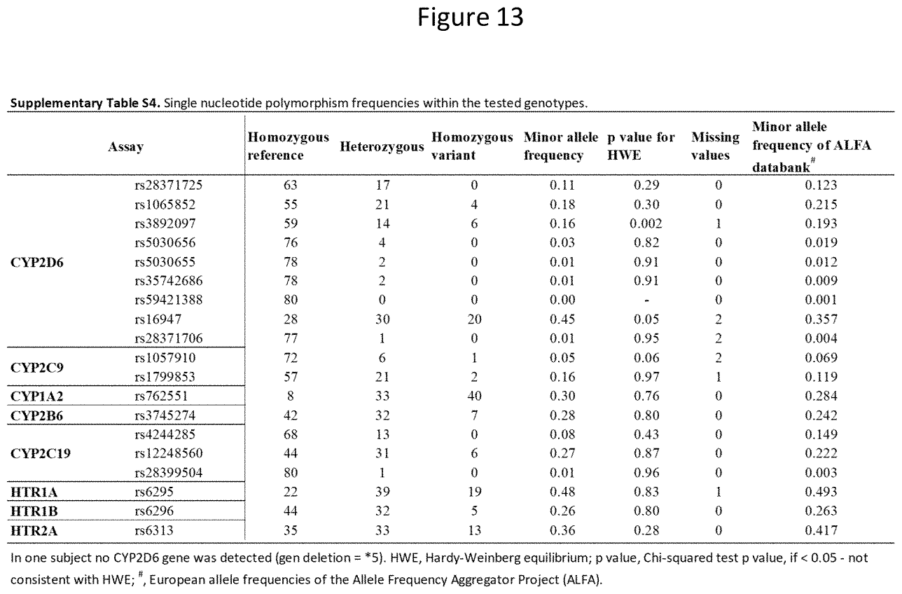

Genomic DNA was extracted from whole blood using the QIAamp DNA Blood Mini Kit (Qiagen, Hombrechtikon, Switzerland) and automated QIAcube system. SNP genotyping was performed using commercial TaqMan SNP genotyping assays (LuBio Science, Lucerne, Switzerland). The following SNPs and respective alleles were assayed: HTR1A rs6295 (assay: C_11904666_10), HTR1B rs9296 (assay: C_2523534_20), HTR2A rs6313 (assay: C_3042197_1_), CYP1A2*1F rs762551 (assay: C_8881221_40), CYP2B6 rs3745274 (assay: C_7817765_60), CYP2C9*2 (rs1799853, assay: C_25625805_10), CYP2C9*3 (rs1057910, assay: C_27104892_10), CYP2C19*2 rs4244285 (assay: C_25986767_70), CYP2C19*4 (rs28399504, assay: C_30634136_10), CYP2C19*17 (rs12248560, assay: C_469857_10), CYP2D6*3 (rs35742686, assay: C_32407232_50), CYP2D6*4 (rs3892097, assay: C_27102431_D0, and rs1065852, assay: C_11484460_40), CYP2D6*6 (rs5030655, assay: C_32407243_20), CYP2D6*9 (rs5030656, assay: C_32407229_60), CYP2D610 (rs1065852), CYP2D617 (rs28371706, assay: C_2222771_A0, and rs16947, assay: C_27102425_10), CYP2D6*29 (rs59421388, assay: C_3486113_20), and CYP2D6*41 (rs28371725, assay: C_34816116_20, and rs16947). CYP2D6 gene deletion (allele *5) and duplication/multiplication (allele *xN) were determined using a TaqMan Copy Number Assay (Hs04502391_cn). Activity scores for CYP2D6 were assigned according to established guidelines (Caudle et al., 2020; Crews et al., 2012; Gaedigk et al., 2008; Hicks et al., 2015; Hicks et al., 2013). To see a distinct effect of CYP2D6 functionality on the pharmacokinetic and pharmacodynamic effects of LSD, the subjects were classified as non-functional CYP2D6 (PMs, activity score=0) and functional CYP2D6 (activity score>0). The activity score for CYP2C9 was generated using the relative metabolic activity of warfarin (Gage et al., 2008; Hashimoto et al., 1996). The genetically determined CYP1A2 activity inducibility was combined with the smoking status of the subject (>5 cigarettes per day=smoker; rs762551 AA=inducible) (Sachse et al., 1999; Vizeli et al., 2017). Predicted CYP2C19 intermediate metabolizers (IMs) included CYP2C191/*2 and CYP2C19*2/17, extensive metabolizers (EMs) included CYP2C191/1, and UMs included both CYP2C1917/17 and CYP2C191/17 (Hicks et al., 2013). No CYP2C19 PM was identified within the sample. For CYP2B6, the reduced-activity SNP rs3745274 (516G>T, CYP2B6*6 or CYP2B6*9, assay: C_7817765_60) was determined. Allele frequencies for the classification of CYP2D6 and CYP2C9 are shown in FIGS. 11 and 12 (Tables S2 and S3), respectively. All tested SNP frequencies are comparable to the Allele Frequency Aggregator (ALFA) Project databank and are listed in FIG. 13 (Table S4) (L. Phan, 2020).

Statistical Analysis

All data were analyzed using the R language and environment for statistical computing (R Core Team, 2019). To test for genotype effects, the pharmacokinetic parameters or effects of LSD (Δ LSD-placebo) were compared using one-way analysis of variance (ANOVA) with genotype as the between-group factor. The data is shown as actual values and z-scores per study because the actual values may be biased by a possible unequal distribution of genotypes across studies.

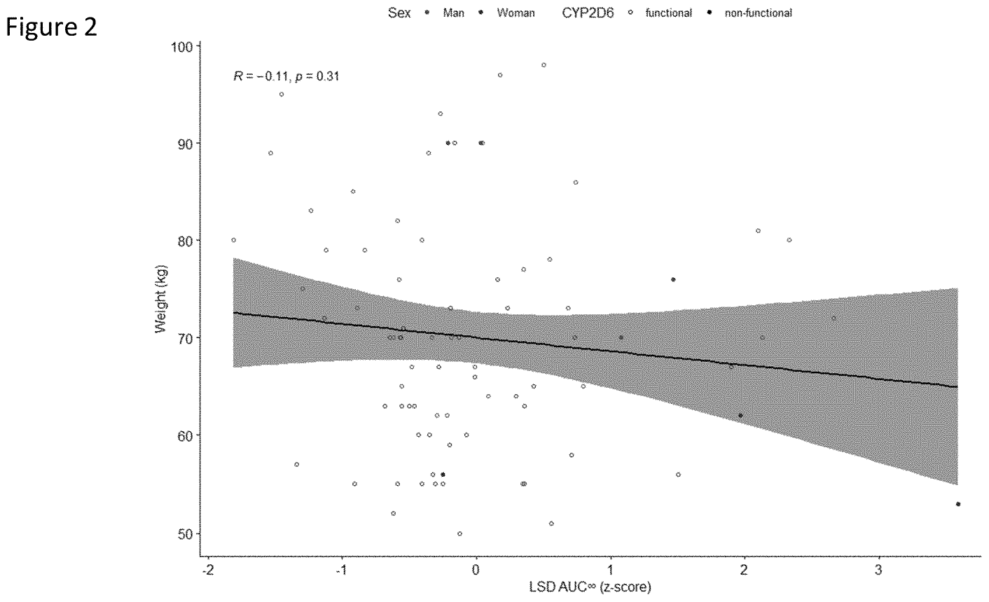

The statistics were not corrected for sex or bodyweight because no correlations were found between sex or bodyweight and exposure to the drug (LSD AUC∞) (FIG. 2, 51). As shown in FIG. 2, an outlying individual was identified as non-functional CYP2D6. To minimize the effect of outliers and associated non-normal data distributions on the parametric statistics, the results were confirmed for the influence of CYP2D6 functionality on the pharmacokinetics and effects of LSD with nonparametric statistics (Wilcoxon signed-rank test and Kruskal-Wallis test). The LSD AUC∞ values were z-normalized per study. Dot colors indicate male (dark-blue) or female (red) participants. Filled dot indicates a non-functional CYP2D6 genotype. Sex or body weight had no relevant effect on the concentration of LSD in plasma.

The level of significance was set at p<0.05. P-values in pharmacokinetic analysis were not corrected for multiple testing because hypotheses for the influence of certain enzyme activities (i.e., CYP2D6) were made a priori. For the analysis of the serotonin receptor SNPs (rs6295, rs6296, and rs6313), the primary analysis was performed using an additive genotype model for SNPs. Recessive or dominant model analysis was performed, the results of which are reported only when the additive model was significant. In the serotonin receptor genotype analyses, differences in plasma concentrations of LSD that may be caused by differences in metabolizing enzymes were accounted for by including the LSD AUC∞ z-score as a covariate.

Results

LSD produced significant acute subjective effects on all scales and moderately increased blood pressure, heart rate, and body temperature compared to placebo (FIG. 14, Table S5). Sex or differences in body weight did not relevantly alter the pharmacokinetics of effects of LSD (FIG. 2).

Effects of CYP Genotype on LSD Pharmacokinetics and Acute Effects

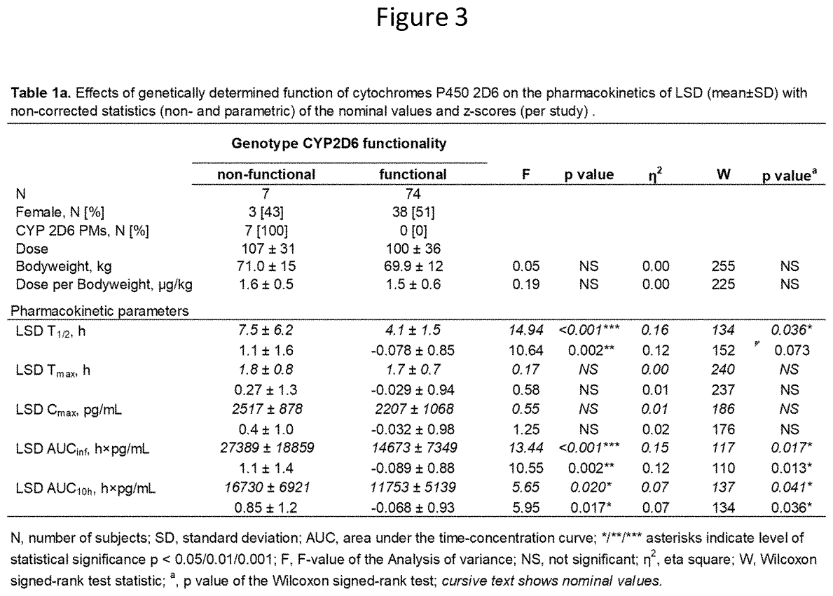

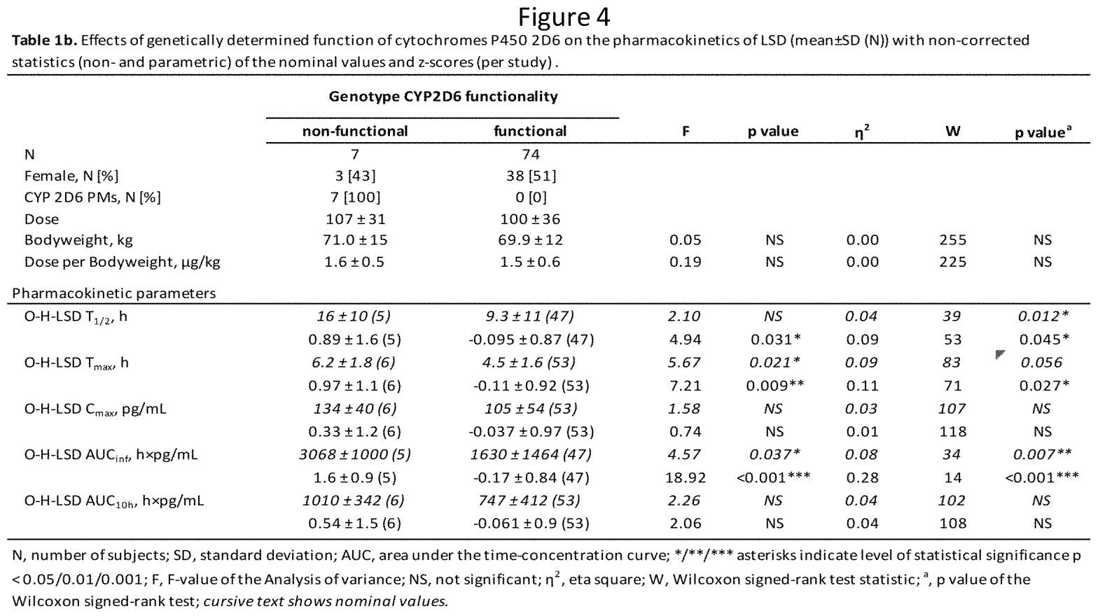

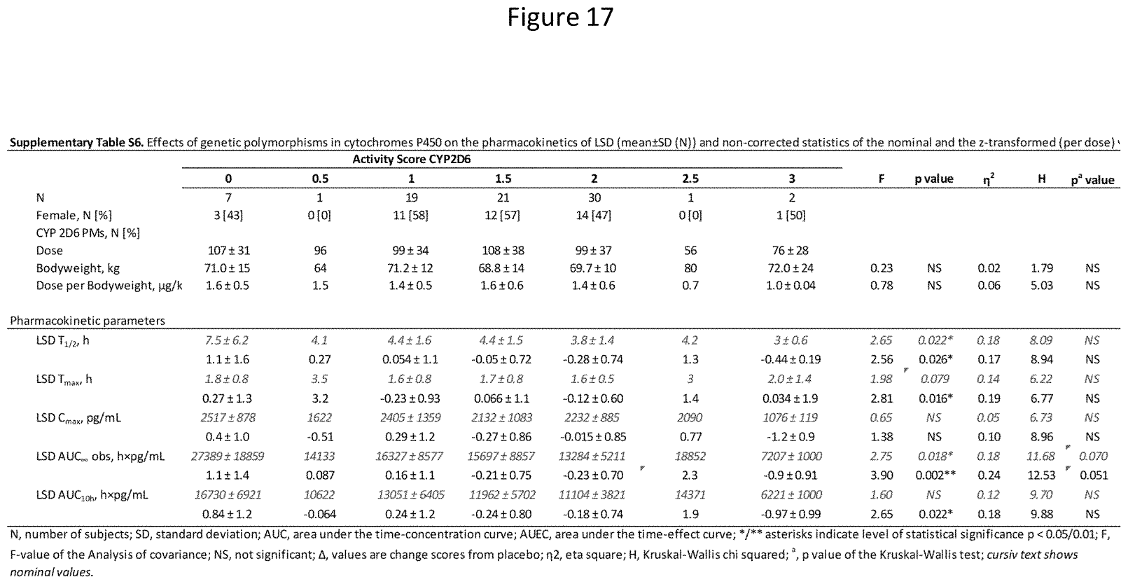

CYP2D6 function significantly influenced the pharmacokinetics and acute effects of LSD (FIGS. 3-5, Table 1a-c and FIG. 1). Specifically, subjects genetically classified as CYP2D6 PMs (non-functional) showed higher exposure to LSD in plasma (FIG. 1) as statistically evidenced by significantly larger AUC∞ and AUC₁₀ values compared with functional CYP2D6 carriers (FIG. 3, Table 1a). In FIG. 1, the shaded area marks the standard error of the mean. CYP2D6 non-functional (N=7) and functional (N=74) subjects received a dose of (mean±SD) 100±30 μg LSD and 98±35 μg LSD, respectively. Both the half-live and AUC values were significantly increased in subjects with non-functional compared with functional CYP2D6 enzymes. Additionally, CYP2D6 PMs also had longer T_{1/2} values consistent with slowed metabolism compared to functional CYP2D6 subjects (FIG. 3, Table 1a) while Cₘₐₓ of LSD was not significantly affected. Furthermore, O—H-LSD AUC∞ values were larger in CYP2D6 PMs compared with functional CYP2D6 subjects (FIG. 4, Table 1b), in parallel with the effects on LSD concentrations and indicating that the conversion to O-H-LSD is independent of CYP2D6. Compartmental modeling for a 100 μg LSD dose administration showed LSD AUC∞ and Cₘₐₓ values for CYP2D6 PMs vs. functional subjects of 24169±13112 vs. 13819±6281 μg/mL*h (F_{1,79}=13.8; p<0.001) and 2369±891 vs. 2061±999 μg/mL (F_{1,79}=0.62; p=0.43), respectively (FIG. 1). Lower CYP2D6 activity was also associated with significantly higher exposure to LSD when analyzed across all CYP2D6 genotype activity score groups (FIG. 17, Table S6).

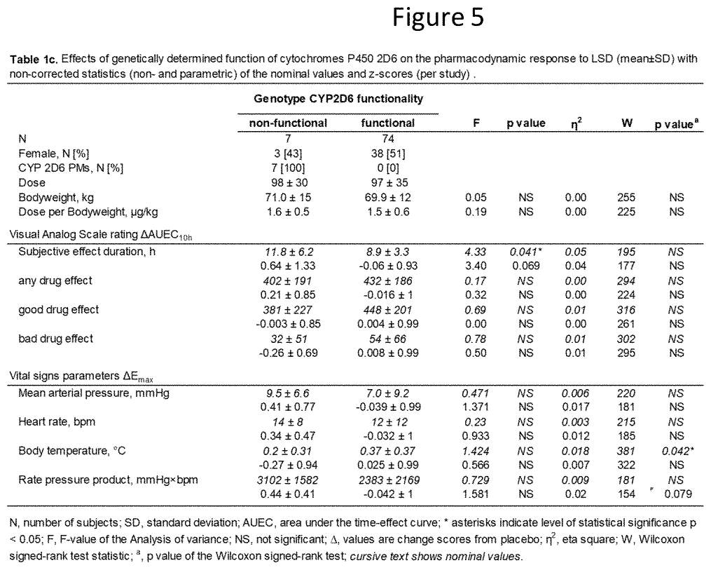

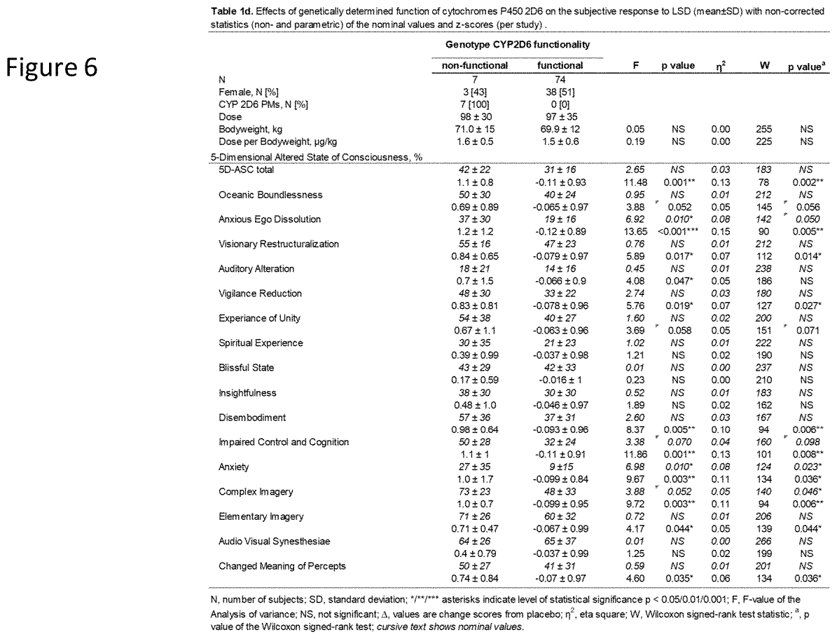

Consistent with the effect on the pharmacokinetic of LSD (FIG. 1), CYP2D6 PMs exhibited a substantially longer duration of the acute subjective response to LSD (FIG. 5, Table 1c) and significantly greater alterations of the mind compared with functional CYP2D6 subjects (FIG. 6, Table 1d). Specifically, ratings on the 5D-ASC total, AED subscale (including disembodiment, impaired control and cognition, and anxiety), and VR subscale (including complex and elementary imagery and changed meaning of percepts) were significantly increased in PMs compared with functional CYP2D6 subjects (FIG. 6, Table 1d). CYP2D6 genotype had no relevant effect on the autonomic response to LSD (FIG. 5, Table 1c).

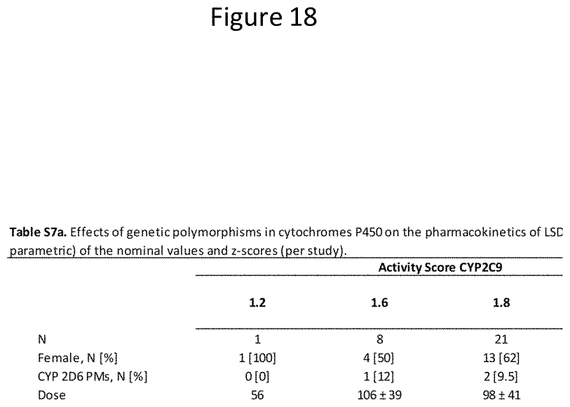

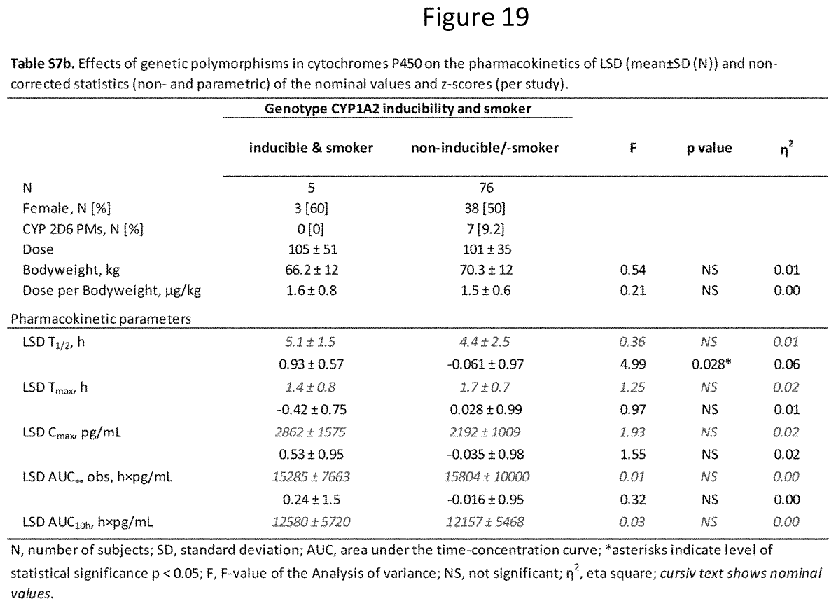

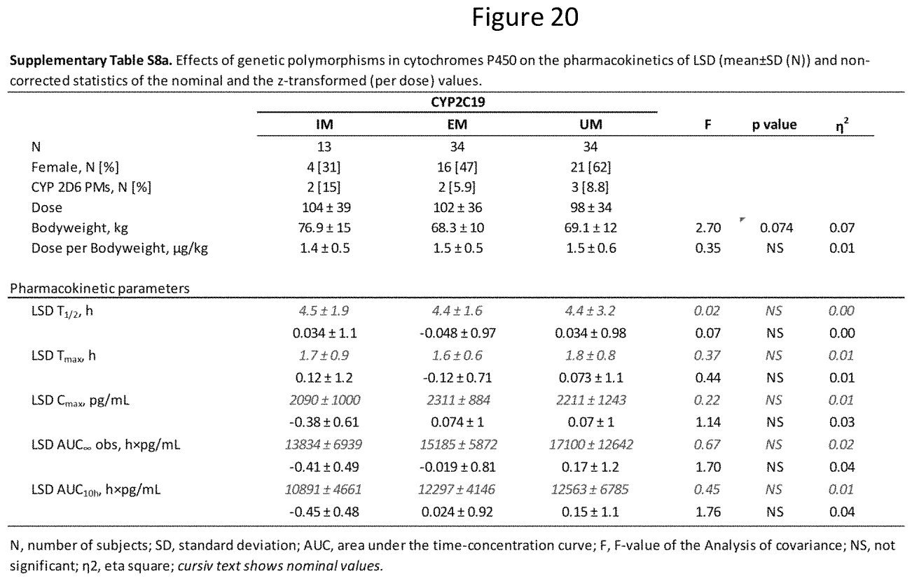

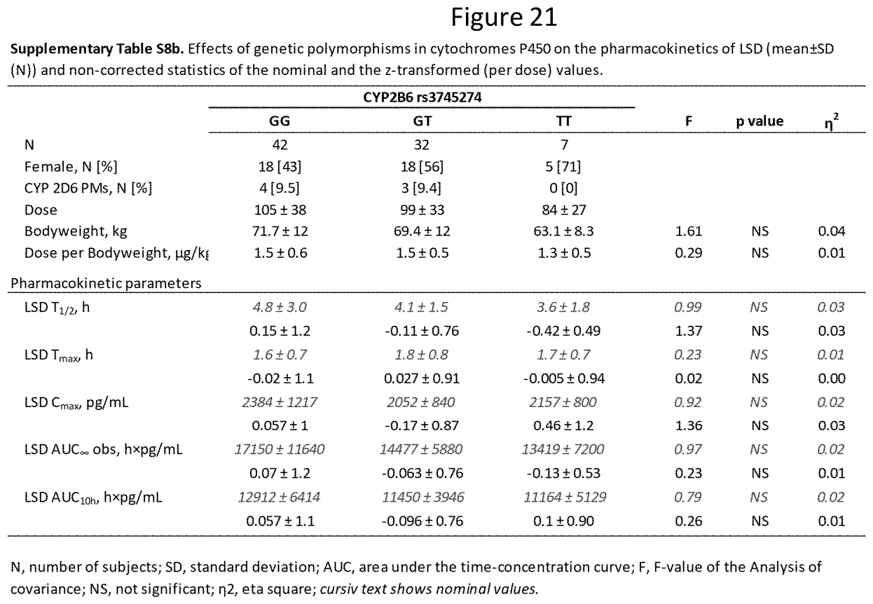

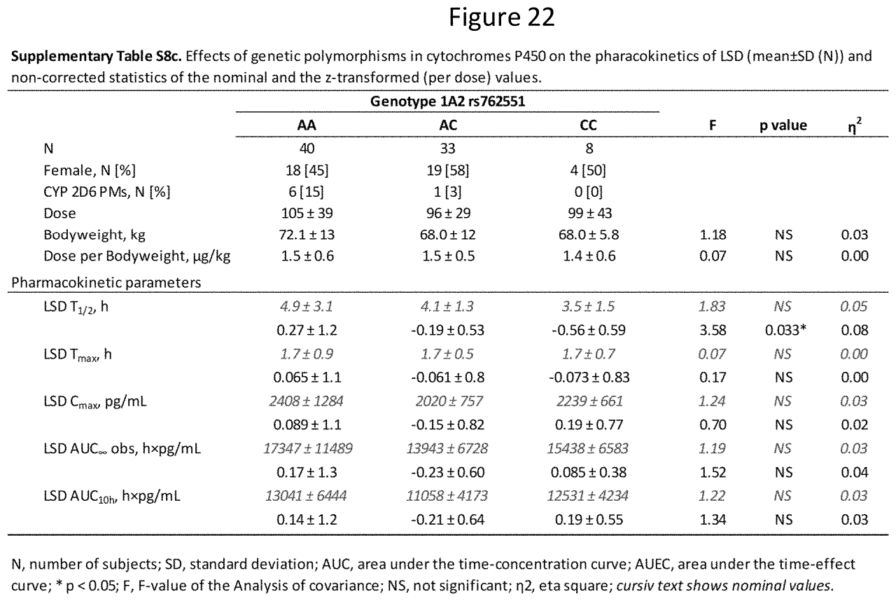

In contrast to CYP2D6, genetic polymorphisms of other CYPs including CYP1A2, CYP2B6, CYP2C19, and CYP2C9 had no relevant effect on the pharmacokinetics or subjective or autonomic effects of LSD (FIG. 17-22, Tables S7a-b and S8a-c).

Effect of 5-HT Receptor Genotype on the Response to LSD

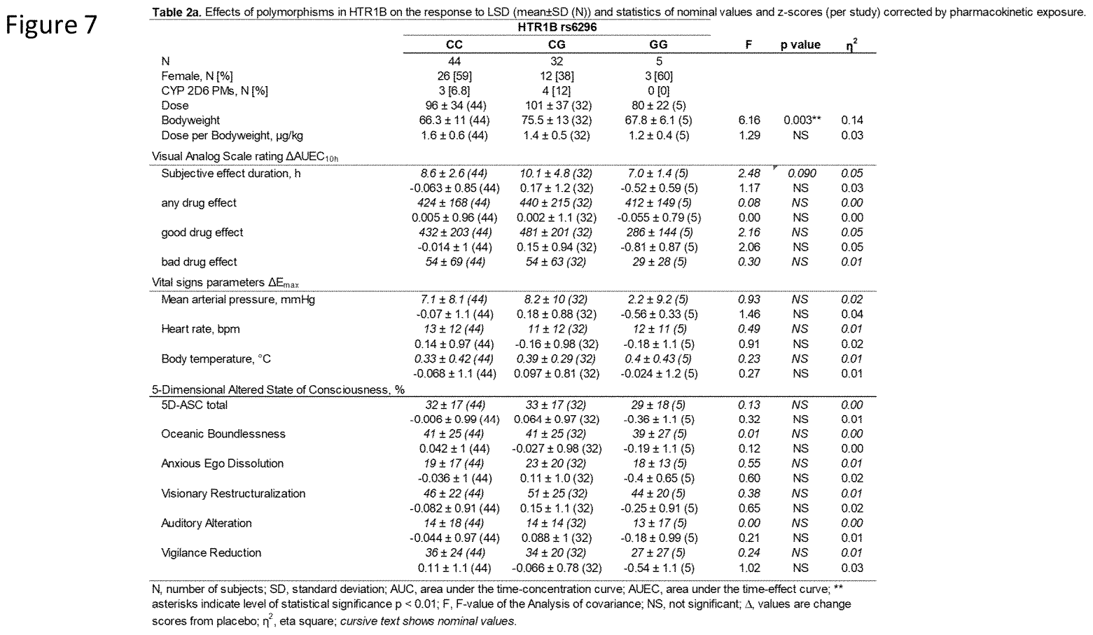

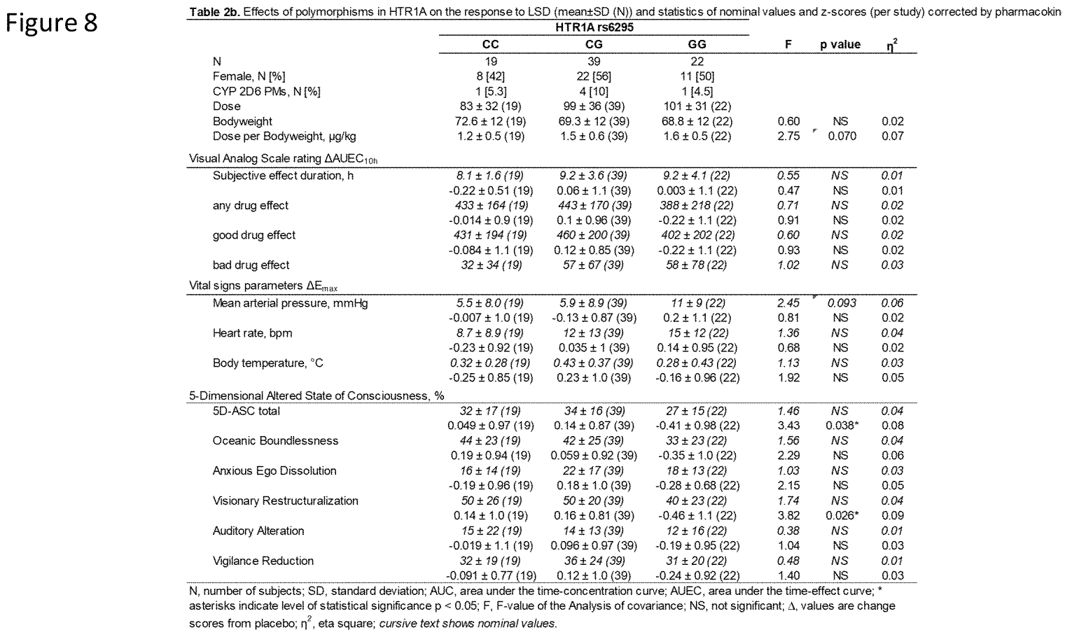

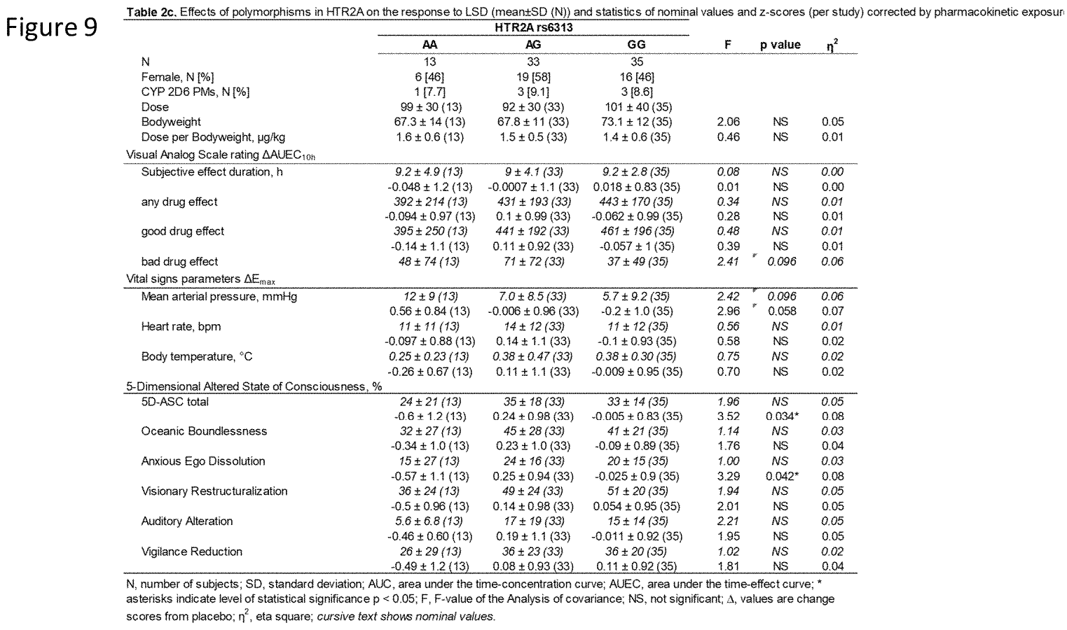

FIG. 7-9 (Table 2a-c) show the effects of polymorphisms in 5-HT receptor genes (HTR1A, HTR1B, and HTR2A) on the acute subjective and autonomic response to LSD. 5-HT receptor gene polymorphisms showed a small effect on the 5D-ASC i.e., HTR2A rs6313 and HTR1A rs6295. Carriers of two HTR2A rs6313 A alleles had lower ratings in the 5D-ASC total score (F_{1,78}=5.88, p<0.05) and AED subscale than G allele carriers (F_{1,78}=5.16, p<0.05). Homozygous carriers of the HTR1A rs6295 G allele rated lower on the 5D-ASC total score and VR subscale than carriers of a C allele (F_{1,78}=6.87, p<0.05 and F_{1,78}=7.75, p<0.01, respectively). Vital parameters were not affected by any of the genotypes studied here.

Interpretation of Study Results

This is the first analysis examining of the influence of genetic polymorphisms on the pharmacokinetics and acute effects of LSD in humans.

The main finding was that genetic polymorphisms of CYP2D6 significantly influenced the pharmacokinetic and subsequently subjective effects of LSD. This allows the novel use of testing of CYP2D6 genes to predict an ideal dose of LSD in an individual and to reduce an overdose and associated adverse effects such as anxiety.

Additionally, common mutations in the 5-HT receptor genes also weakly influenced the acute alterations of the mind induced by LSD allowing to further or separately define ideal doses of LSD in an individual. However, the impact and extent of this effect moderation is weaker than that of the CYP2D6 gene.

LSD is metabolized almost completely in the human body and only small amounts of the parent drug (˜1%) are excreted in urine (Dolder et al., 2015). In vitro studies in human liver microsomes and human liver S9 fraction indicated a role for CYP enzymes in the metabolism of LSD (Luethi et al., 2019; Wagmann et al., 2019). Specifically, CYP2D6 is involved in the N-demethylation of LSD to nor-LSD (Luethi et al., 2019). The present study provides novel in vivo evidence that CYP2D6 is involved in the metabolism of LSD in humans and specifically that genetic polymorphisms influence both the metabolism and the acute response to LSD in humans. Plasma nor-LSD concentrations in humans are mostly too low to be measured even with highly sensitive methods (Steuer et al., 2017). However, an increase was found in both LSD and O-H-LSD plasma concentrations in individuals with a non-functional CYP2D6 genotype consistent with a role of CYP2D6 in the formation of nor-LSD but not O-H-LSD. Thus, CYP2D6 is a crucial player in the degradation of LSD, but not in the formation of its main metabolite 0-H-LSD.

The role of CYP2D6 can be further be investigated in drug-drug interaction studies using LSD with and without selective CYP2D6 inhibition. This is also of interest because LSD can be therapeutically used in patients with psychiatric disorders and using a serotonin reuptake inhibitor treatment, which can also act as CYP2D6 inhibitors (mostly fluoxetine and paroxetine). Accordingly, the present invention can be further refined by adding information on co-use of medications with CYP2D6 inhibiting or inducing potential within algorithms or by those skilled in the art applying the present invention.

As for other CYP enzymes, CYP2C19 was involved in the formation of nor-LSD in vitro (Wagmann et al., 2019). However, no influence was found of its genotype on the pharmacokinetics of LSD in the present study and no adjustment of dose of LSD appears to be needed.

Furthermore, CYP2C9 and CYP1A2 were reported to contribute to the hydroxylation of LSD to O—H-LSD (Luethi et al., 2019; Wagmann et al., 2019). CYP2C9 also catalyzes the N-deethylation to lysergic acid monoethylamide (LAE) (Wagmann et al., 2019). However, no effects of the CYP2C9 genotype on the pharmacokinetics of LSD were observed in the present study in humans. As for CYP1A2, no common loss-of-function polymorphisms have been identified to date. However, CYP1A2 is inducible by tobacco smoking in subjects with the common SNP rs762551 A/A genotype compared with the C/A and C/C genotypes (Sachse et al., 1999). Accordingly, CYP1A2 activity inducibility was combined with the smoking status of the subject (>5 cigarettes per day=smoker). In a similar pharmacogenetic study with MDMA, higher MDA levels (the minor metabolite of MDMA) were found in subjects who smoked 6-10 cigarettes a day and possessed the inducible genotype of the CYP1A2 compared with subjects who smoked less and/or had the non-inducible polymorphism (Vizeli et al., 2017). An influence of the CYP1A2 genotype/smokers status was not found on the pharmacokinetic of LSD in the current study. However, there were only five subjects enrolled in the present study who met both requirements of being a smoker and possessing an inducible CYP1A2 genotype. Thus, the present data indicates no adjustment of dose of LSD based on CYP1A2 genotype.

The pharmacogenetic influence of metabolizing enzymes on LSD appears quite similar to MDMA. For both psychoactive substances, LSD and MDMA, only polymorphisms in CYP2D6 seem to substantially impact pharmacokinetics and subjective effects (Vizeli et al., 2017). However, because MDMA inhibits CYP2D6 and its own metabolism (i.e., autoinhibition), the effect of CYP2D6 genotype variations is limited and evident only during the onset of the MDMA effects during the first 2 hours after administration (Schmid et al., 2016).

In contrast, for LSD, CYP2D6 genotype moderation appears to become more relevant later on during the elimination phase and increasing the AUC and half-life of LSD and its duration of effect rather than absorption and the early effect peak. CYP2D6 PMs showed approximately 75% more total drug exposure (greater AUC values) than individuals with a functional CYP2D6 enzyme. There was only a non-significant approximately 15% higher mean peak concentration. Therefore, the total drug exposure, which is reflected by the AUC∞, was mainly determined by the reduced elimination after the peak. This pattern can also be seen with the subjective effects of LSD. While the VASs peak effects were not different between the different CYP genotypes, the 5D-ASC ratings that reflect subjective alterations of the mind over the entire day showed distinct differences depending on CYP2D6 functionality. The non-functional CYP2D6 group reported an overall more altered state of consciousness with particularly higher ratings of Disembodiment, Impaired Control and Cognition, Anxiety, Complex Imagery, Elementary Imagery, and Changed Meaning of Percepts.

The genetic effects on the acute subjective response to LSD is clinically relevant and the present invention is therefore practically useful and effective to adjust the dose and partly solve the problem of overdosing in vulnerable subject.

Several studies in healthy subjects and patients found associations between the extent and quality of the acute subjective experience and the long-term effects of psychedelics including LSD (Griffiths et al., 2008; Griffiths et al., 2016; Roseman et al., 2017; Ross et al., 2016; Schmid & Liechti, 2018). Typically, greater substance-induced OB and more mystical-type effects could be associated with more beneficial long-term effects. Specifically with regard to the 5D-ASC rating scale used in the present analysis, greater acutely psilocybin-induced OB and lower AED scores predicted better therapeutic outcomes at 5 weeks in patients with depression while VR scores had no significant effects (Roseman et al., 2017).

There was an identical prediction pattern for acute responses to LSD (200 μg) with positive OB, negative AED and no VR score associations with beneficial effects on depression, anxiety and overall psychological distress 2 or 5 weeks after LSD administration in patients with anxiety disorder (Liechti personal communication).

Considering that CYP2D6 PMs mainly showed greater LSD-induces ratings on AED and VR but not OB scores, these subjects are expected to have an overall more challenging acute experience with namely more acute anxiety and possibly reduced therapeutic effects.

The present invention including genotyping is expected to be particularly useful in patients who undergo LSD-assisted therapy. Based on the present findings CYP2D6 PMs can be expected to benefit from approximately 50% lower doses than those that are used in functional CYP2D6 individuals. This direct consequence based on the present data and approach is in line with the observation that higher doses of 200 μg LSD compared to 100 μg did not result in higher OB ratings but increased AED and anxiety on the 5D-ASC (Holze et al., 2021b).

The present invention can require some modifications as it is further developed and along its implementation. Even though developed using the largest available sample of healthy human subjects who received LSD in placebo-controlled studies, the sample size is still relatively small. Although the sample size was sufficient to detect an effect of functionally very different genotypes (i.e., CYP2D6), the sample used to develop the invention may have been too small to detect smaller effect moderations.

In addition, CYP3A4 can play a role in the metabolism of LSD but polymorphisms are rare (Werk & Cascorbi, 2014). Thus, for CYP3A4 genotyping is not useful but phenotyping could be used and added as a modification or extension to the present invention.

The present invention is also useful when considering drug-drug interactions between concomitantly used medications and LSD. CYP2D6 inhibitors should be stopped and allowing sufficient time for the enzyme to regenerate (up to two weeks) before LSD is used. Alternatively, in the presence of CYP2D6 inhibitors the dose of LSD should be reduced by 50% based on the findings of the present invention.

To conclude, this is the first study examining the influence of genetic polymorphisms on the pharmacokinetics and acute effects of LSD in humans. Genetic polymorphisms of CYP2D6 had a significant influence on the pharmacokinetic and subsequently on the subjective effects of LSD. No effect on the pharmacokinetics or response to LSD was observed with other CYPs. Additionally, common mutations in the 5-HT receptor genes weakly moderated the subjective effect of LSD.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Caudle K E, Sangkuhl K, Whirl-Carrillo M, Swen J J, Haidar C E, Klein T E, Gammal R S, Relling M V, Scott S A, Hertz D L, Guchelaar H J, & Gaedigk A (2020). Standardizing CYP2D6 Genotype to Phenotype Translation: Consensus Recommendations from the Clinical Pharmacogenetics Implementation Consortium and Dutch Pharmacogenetics Working Group. Clin Transl Sci 13: 116-124.
2. Crews K R, Gaedigk A, Dunnenberger H M, Klein T E, Shen D D, Callaghan J T, Kharasch E D, Skaar T C, & Clinical Pharmacogenetics Implementation C (2012). Clinical Pharmacogenetics Implementation Consortium (CPIC) guidelines for codeine therapy in the context of cytochrome P450 2D6 (CYP2D6) genotype. Clin Pharmacol Ther 91: 321-326.
3. Dittrich A (1998). The standardized psychometric assessment of altered states of consciousness (ASCs) in humans. Pharmacopsychiatry 31 (Suppl 2): 80-84.
4. Dolder P C, Holze F, Liakoni E, Harder S, Schmid Y, & Liechti M E (2017a). Alcohol acutely enhances decoding of positive emotions and emotional concern for positive stimuli and facilitates the viewing of sexual images. Psychopharmacology 234: 41-51.
5. Dolder P C, Schmid Y, Haschke M, Rentsch K M, & Liechti M E (2015). Pharmacokinetics and concentration-effect relationship of oral LSD in humans. Int J Neuropsychopharmacol 19: pyv072.
6. Dolder P C, Schmid Y, Steuer A E, Kraemer T, Rentsch K M, Hammann F, & Liechti M E (2017b). Pharmacokinetics and pharmacodynamics of lysergic acid diethylamide in healthy subjects. Clin Pharmacokinetics 56: 1219-1230.
7. Eshleman A J, Wolfrum K M, Reed J F, Kim S O, Johnson R A, & Janowsky A (2018). Neurochemical pharmacology of psychoactive substituted N-benzylphenethylamines: High potency agonists at 5-HT2A receptors. Biochem Pharmacol 158: 27-34.
8. Gaedigk A (2013). Complexities of CYP2D6 gene analysis and interpretation. Int Rev Psychiatry 25: 534-553.
9. Gaedigk A, Simon S D, Pearce R E, Bradford L D, Kennedy M J, & Leeder J S (2008). The CYP2D6 activity score: translating genotype information into a qualitative measure of phenotype. Clin Pharmacol Ther 83: 234-242.
10. Gage B F, Eby C, Johnson J A, Deych E, Rieder M J, Ridker P M, Milligan P E, Grice G, Lenzini P, Rettie A E, Aquilante C L, Grosso L, Marsh S, Langaee T, Farnett L E, Voora D, Veenstra D L, Glynn R J, Barrett A, & McLeod H L (2008). Use of pharmacogenetic and clinical factors to predict the therapeutic dose of warfarin. Clin Pharmacol Ther 84: 326-331.
11. Garcia-Romeu A, Griffiths R R, & Johnson M W (2014). Psilocybin-occasioned mystical experiences in the treatment of tobacco addiction. Curr Drug Abuse Rev 7: 157-164.
12. Ghasemi A, Seifi M, Baybordi F, Danaei N, & Samadi Rad B (2018). Association between serotonin 2A receptor genetic variations, stressful life events and suicide. Gene 658: 191-197.
13. Gong P, Liu J, Blue P R, Li S, & Zhou X (2015). Serotonin receptor gene (HTR2A) T102C polymorphism modulates individuals' perspective taking ability and autistic-like traits. Front Hum Neurosci 9: 575.
14. Griffiths R, Richards W, Johnson M, McCann U, & Jesse R (2008). Mystical-type experiences occasioned by psilocybin mediate the attribution of personal meaning and spiritual significance 14 months later. J Psychopharmacol 22: 621-632.
15. Griffiths R R, Johnson M W, Carducci M A, Umbricht A, Richards W A, Richards B D, Cosimano M P, & Klinedinst M A (2016). Psilocybin produces substantial and sustained decreases in depression and anxiety in patients with life-threatening cancer: a randomized double-blind trial. J Psychopharmacol 30: 1181-1197.
16. Hakulinen C, Jokela M, Hintsanen M, Merjonen P, Pulkki-Raback L, Seppala I, Lyytikainen L P, Lehtimaki T, Kahonen M, Viikari J, Raitakari O T, & Keltikangas-Jarvinen L (2013). Serotonin receptor 1B genotype and hostility, anger and aggressive behavior through the lifespan: the Young Finns study. J Behav Med 36: 583-590.
17. Hashimoto Y, Otsuki Y, Odani A, Takano M, Hattori H, Furusho K, & Iui K (1996). Effect of CYP2C polymorphisms on the pharmacokinetics of phenytoin in Japanese patients with epilepsy. Biol Pharm Bull 19: 1103-1105.
18. Hasler F, Grimberg U, Benz M A, Huber T, & Vollenweider F X (2004). Acute psychological and physiological effects of psilocybin in healthy humans: a double-blind, placebo-controlled dose-effect study. Psychopharmacology 172: 145-156.
19. Hicks J K, Bishop J R, Sangkuhl K, Muller D J, Ji Y, Leckband S G, Leeder J S, Graham R L, Chiulli D L, A L L, Skaar T C, Scott S A, Stingl J C, Klein T E, Caudle K E, & Gaedigk A (2015). Clinical pharmacogenetics implementation consortium (CPIC) guideline for CYP2D6 and CYP2C19 genotypes and dosing of selective serotonin reuptake inhibitors. Clin Pharmacol Ther 98: 127-134.
20. Hicks J K, Swen J J, Thorn C F, Sangkuhl K, Kharasch E D, Ellingrod V L, Skaar T C, Muller D J, Gaedigk A, Stingl J C, & Clinical Pharmacogenetics Implementation C (2013). Clinical pharmacogenetics implementation consortium guideline for CYP2D6 and CYP2C19 genotypes and dosing of tricyclic antidepressants. Clin Pharmacol Ther 93: 402-408.
21. Hintzen A, & Passie T (2010) The pharmacology of LSD: a critical review. Oxford University Press: Oxford.
22. Holze F, Duthaler U, Vizeli P, Muller F, Borgwardt S, & Liechti M E (2019). Pharmacokinetics and subjective effects of a novel oral LSD formulation in healthy subjects. Br J Clin Pharmacol 85: 1474-1483.
23. Holze F, Liechti M E, Hutten N, Mason N L, Dolder P C, Theunissen E L, Duthaler U, Feilding A, Ramaekers J G, & Kuypers K P C (2021a). Pharmacokinetics and pharmacodynamics of lysergic acid diethylamide microdoses in healthy participants. Clin Pharmacol Ther 109: 658-666.
24. Holze F, Vizeli P, Ley L, Muller F, Dolder P, Stocker M, Duthaler U, Varghese N, Eckert A, Borgwardt S, & Liechti M E (2021b). Acute dose-dependent effects of lysergic acid diethylamide in a double-blind placebo-controlled study in healthy subjects. Neuropsychopharmacology 46: 537-544.
25. Holze F, Vizeli P, Muller F, Ley L, Duerig R, Varghese N, Eckert A, Borgwardt S, & Liechti M E (2020). Distinct acute effects of LSD, MDMA, and D-amphetamine in healthy subjects. Neuropsychopharmacology 45: 462-471.
26. Houston J P, Zou W, Aris V, Fijal B, Chen P, Heinloth A N, & Martinez J (2012). Evaluation of genetic models for response in a randomized clinical trial of duloxetine in major depressive disorder. Psychiatry Res 200: 63-65.
27. Huang Y Y, Battistuzzi C, Oquendo M A, Harkavy-Friedman J, Greenhill L, Zalsman G, Brodsky B, Arango V, Brent D A, & Mann J J (2004). Human 5-HT1A receptor C(−1019)G polymorphism and psychopathology. Int J Neuropsychopharmacol 7: 441-451.
28. Kim K, Che T, Panova O, DiBerto J F, Lyu J, Krumm B E, Wacker D, Robertson M J, Seven A B, Nichols D E, Shoichet B K, Skiniotis G, & Roth B L (2020). Structure of a Hallucinogen-Activated Gq-Coupled 5-HT2A Serotonin Receptor. Cell 182: 1574-1588 e1519.
29. Kraehenmann R, Pokorny D, Aicher H, Preller K H, Pokorny T, Bosch O G, Seifritz E, & Vollenweider F X (2017). LSD Increases Primary Process Thinking via Serotonin 2A Receptor Activation. Front Pharmacol 8: 814.
30. Phan Y J, H. Zhang, W. Qiang, E. Shekhtman, D. Shao, D. Revoe, R. Villamarin, E. Ivanchenko, M. Kimura, Z. Y. Wang, L. Hao, N. Sharopova, M. Bihan, A. Sturcke, M. Lee, N. Popova, W. Wu, C. Bastiani, M. Ward, J. B. Holmes, V. Lyoshin, K. Kaur, E. Moyer, M. Feolo, and B. L. Kattman. (2020). ALFA: Allele Frequency Aggregator. National Center for Biotechnology Information: U.S. National Library of Medicine.
31. Liechti M E (2017). Modern clinical research on LSD. Neuropsychopharmacology 42: 2114-2127.
32. Luethi D, Hoener M C, Krahenbuhl S, Liechti M E, & Duthaler U (2019). Cytochrome P450 enzymes contribute to the metabolism of LSD to nor-LSD and 2-oxo-3-hydroxy-LSD: Implications for clinical LSD use. Biochem Pharmacol 164: 129-138.
33. Nichols D E (2016). Psychedelics. Pharmacol Rev 68: 264-355.
34. Nichols D E, & Grob C S (2018). Is LSD toxic? Forensic Sci Int 284: 141-145.
35. Passie T, Halpern J H, Stichtenoth D O, Emrich H M, & Hintzen A (2008). The pharmacology of lysergic acid diethylamide: a review. CNS Neurosci Ther 14: 295-314.
36. Polesskaya O O, Aston C, & Sokolov B P (2006). Allele C-specific methylation of the 5-HT2A receptor gene: evidence for correlation with its expression and expression of DNA methylase DNMT1. J Neurosci Res 83: 362-373.
37. Preissner S C, Hoffmann M F, Preissner R, Dunkel M, Gewiess A, & Preissner S (2013). Polymorphic cytochrome P450 enzymes (CYPs) and their role in personalized therapy. PLoS One 8: e82562.
38. Preller K H, Herdener M, Pokorny T, Planzer A, Kraehenmann R, Stampfli P, Liechti M E, Seifritz E, & Vollenweider F X (2017). The fabric of meaning and subjective effects in LSD-induced states depend on serotonin 2A receptor activation Curr Biol 27: 451-457.
39. R Core Team (2019). R: A language and environment for statistical computing.
40. Rickli A, Moning O D, Hoener M C, & Liechti M E (2016). Receptor interaction profiles of novel psychoactive tryptamines compared with classic hallucinogens. Eur Neuropsychopharmacol 26: 1327-1337.
41. Roseman L, Nutt D J, & Carhart-Harris R L (2017). Quality of acute psychedelic experience predicts therapeutic efficacy of psilocybin for treatment-resistant depression. Front Pharmacol 8: 974.
42. Ross S, Bossis A, Guss J, Agin-Liebes G, Malone T, Cohen B, Mennenga S E, Belser A, Kalliontzi K, Babb J, Su Z, Corby P, & Schmidt B L (2016). Rapid and sustained symptom reduction following psilocybin treatment for anxiety and depression in patients with life-threatening cancer: a randomized controlled trial. J Psychopharmacol 30: 1165-1180.
43. Sachse C, Brockmoller J, Bauer S, & Roots I (1997). Cytochrome P450 2D6 variants in a Caucasian population: allele frequencies and phenotypic consequences. Am J Hum Genet 60: 284-295.
44. Sachse C, Brockmoller J, Bauer S, & Roots I (1999). Functional significance of a C>A polymorphism in intron 1 of the cytochrome P450 CYP1A2 gene tested with caffeine. Br J Clin Pharmacol 47: 445-449.
45. Schmid Y, Enzler F, Gasser P, Grouzmann E, Preller K H, Vollenweider F X, Brenneisen R, Mueller F, Borgwardt S, & Liechti M E (2015). Acute effects of lysergic acid diethylamide in healthy subjects. Biol Psychiatry 78: 544-553.
46. Schmid Y, Gasser P, Oehen P, & Liechti M E (2021). Acute subjective effects in LSD- and MDMA-assisted psychotherapy. J Psychopharmacol 35: 362-374.
47. Schmid Y, & Liechti M E (2018). Long-lasting subjective effects of LSD in normal subjects. Psychopharmacology (Berl) 235: 535-545.
48. Schmid Y, Vizeli P, Hysek C M, Prestin K, Meyer Zu Schwabedissen H E, & Liechti M E (2016). CYP2D6 function moderates the pharmacokinetics and pharmacodynamics of 3,4-methylene-dioxymethamphetamine in a controlled study in healthy individuals. Pharmacogenet Genomics 26: 397-401.
49. Steuer A E, Poetzsch M, Stock L, Eisenbeiss L, Schmid Y, Liechti M E, & Kraemer T (2017). Development and validation of an ultra-fast and sensitive microflow liquid chromatography-tandem mass spectrometry (MFLC-M S/M S) method for quantification of LSD and its metabolites in plasma and application to a controlled LSD administration study in humans. Drug Test Anal 9: 788-797.
50. Studerus E, Gamma A, Kometer M, & Vollenweider F X (2012). Prediction of psilocybin response in healthy volunteers. PLoS One 7: e30800.
51. Studerus E, Gamma A, & Vollenweider F X (2010). Psychometric evaluation of the altered states of consciousness rating scale (OAV). PLoS One 5: e12412.
52. Vizeli P, Meyer Zu Schwabedissen H E, & Liechti M E (2019). Role of Serotonin Transporter and Receptor Gene Variations in the Acute Effects of MDMA in Healthy Subjects. ACS Chem Neurosci 10: 3120-3131.
53. Vizeli P, Schmid Y, Prestin K, Meyer zu Schwabedissen H E, & Liechti M E (2017). Pharmacogenetics of ecstasy: CYP1A2, CYP2C19, and CYP2B6 polymorphisms moderate pharmacokinetics of MDMA in healthy subjects. Eur Neuropsychopharmacol 27: 232-238.
54. Vizeli P, Straumann I, Holze F, Schmid Y, Dolder P C, & Liechti M E (2021). Genetic influence of CYP2D6 on pharmacokinetics and acute subjective effects of LSD in a pooled analysis. Sci Rep 11: 10851.
55. Wacker D, Wang S, McCorvy J D, Betz R M, Venkatakrishnan A J, Levit A, Lansu K, Schools Z L, Che T, Nichols D E, Shoichet B K, Dror R O, & Roth B L (2017). Crystal structure of an LSD-bound human serotonin receptor. Cell 168: 377-389 e312.
56. Wagmann L, Richter L H J, Kehl T, Wack F, Bergstrand M P, Brandt S D, Stratford A, Maurer H H, & Meyer M R (2019). In vitro metabolic fate of nine LSD-based new psychoactive substances and their analytical detectability in different urinary screening procedures. Anal Bioanal Chem 411: 4751-4763.
57. Werk A N, & Cascorbi I (2014). Functional gene variants of CYP3A4. Clin Pharmacol Ther 96: 340-348.

What is claimed is:

1. A method of dosing LSD in treating patients, including the steps of:
assessing genetic characteristics in the patient by identifying polymorphisms of CYP2D6 before use of a composition chosen from the group consisting of LSD, analogs thereof, derivatives thereof, and salts thereof;
administering the composition to the patient based on the patient genetics, wherein a 50% dose is administered in a patient with non-functional CYP2D6 compared to a dose in functional CYP2D6 individuals; and
producing maximum positive subjective acute effects in the patient and/or reducing anxiety and negative effects.

2. The method of claim 1, wherein said assessing step is further defined as identifying 5HTR1A rs6295 and 5HTR2A rs6313 genotypes.

3. A method of determining a preferred dose of LSD, including the steps of:
determining metabolic or/and genetic markers in a patient by assessing CYP2D6 activity in the patient;
adjusting a dose of a composition chosen from the group consisting of LSD, analogs thereof, derivatives thereof, and salts thereof based on metabolic and genetic activity (pharmacogenetics), wherein if CYP26D activity is poor or not present, the dose is adjusted to 50% of a dose with functional CYP26D;
administering the dose of the composition to the patient; and
producing maximum positive subjective acute effects in the patient and/or reducing anxiety and negative effects.

4. The method of claim 3, wherein said determining step further includes assessing 5HTR1A rs6295 and 5HTR2A rs6313 genotypes in a patient.

5. The method of claim 3, wherein the metabolic activity is related to enzymatic digestion.

6. The method of claim 3, wherein pharmacological activity is related to activation or binding to receptors.
